(19) United States
(12) Patent Application Publication
(10) Pub. No.: US 2003/0118661 A1
(43) Pub. Date: Jun. 26, 2003

(54) Pure biological life-prolong product

(76) Inventor: Yongzhong Li, Chengdu City (CN)

Correspondence Address: PALMER & DODGE, LLP, KATHLEEN M. WILLIAMS, 111 HUNTINGTON AVENUE, BOSTON, MA, 02199, US

(21) Appl. No.: 10/193,360

(22) Filed: Jul. 11, 2002

(30) Foreign Application Priority Data
Sep. 3, 2001 (CN) 01128808.6

(51) Int. Cl. A61K 35/78; A61K 35/12; A61K 31/70

(52) U.S. Cl. 424/520; 424/728; 514/23

(57) ABSTRACT

This invention opens a prescription of a pure biological life-prolong product. The product is made from the following ingredients in weight proportion: soft-shelled turtle 100, ageratum 2-5, ginseng 2-5, and glucose 2-5. Every ingredient is ground into powder and mixed together in proportion; then add an appropriate amount of water, boil by slow fire and filtrate into aqua form, or dry the filtrate and extract into powder form, bolus form or tablet form. This medicine can improve human immune functions, accelerate human metabolism by the increase of cell division and cell regeneration so as to reach the goal of cancer prevention and treatment, youth keeping and life prolong.

### BACKGROUND OF INVENTION

1. Field of the Invention

This invention relates to a pure Chinese traditional medicine product made by pure-biological techniques, especially a kind of pure Chinese traditional medicine product for immune function improvement, cancer prevention and treatment, youth keeping and life prolong.

2. Background of the Invention

With the development of science and technology, people understand their vital phenomena deeper and deeper. Invaded by various pathogenic factors, humans become senescent till death. Currently, all kinds of medical drugs aim at prevention or kill of these pathogenic factors. However, on the one hand, these prevented or killed pathogenic factors are still in human bodies, damaging active normal cells. Even after a long time, they still can re-gather and violate human bodies. On the other hand, body cells damaged by the pathogenic factors do not regenerate and new cells cannot supplant dead cells timely. Thus human functions are in long-term abnormal conditions, making not only impaired parts to be damaged continuously but also normal parts to be damaged too. Therefore, to clean up the interior of human bodies and to keep normal function status by promotion of cell division and cell regeneration, are necessary conditions of health and life.

### SUMMARY OF THE INVENTION

The purpose of the invention is to provide a pure Chinese traditional medicine product which can activate human immune system, improve human immune functions, accelerate human metabolism by the increase of cell division and cell regeneration, finally reach the goal of cancer prevention and treatment, youth keeping and life prolong.

### DETAILED DESCRIPTION OF THE INVENTION

The product is made by mixing the following medicines in weight proportion:

| | |
|---|---|
| Soft-shelled turtle | 100 |
| Ageratum | 2-5 |
| Ginseng root | 2-5 |
| Glucose | 2-5 |

Mature soft-shelled turtle, removing its head and neck, keeping its body and tail, is used in this prescription. With salty and mild taste and turquoise color, belonging to Yin (the female or negative principle in human), it has the efficacy of nourishing Yin and removing heat. It can cure diseases with over fatigue, hectic fever, alternate attacks of chills and fever, warm-type malaria or the mother of malaria, waist and flank pain, bloody mass, hemorrhoid, suppression of manner, difficult labor, intestinal abscess, carbuncles or boils, epilepsy due to fright, erythema or poxes, terminal or shrinking Yin, pathological heat accumulated in the blood system. What has green color, nine ribs and a weight of more than 7 liang (50 g) is the top grade. It should be baked with vinegar. If used to treat chronic infectious consumptive diseases, it should be baked with urine of a baby boy or boiled into an ointment. Turtle flesh can cool the blood, nourish Yin and cure malaria. Aversion to aluminite, it counteracts amaranth and eggs. Turtle gall tastes peppery, and can replace capsicum to remove fishy taste.

Ginseng or American ginseng has the efficacy of invigorating original vital energy and purging heat. The uncooked tastes sweet and bitter with a little cold in nature, while the cooked sweet and warm. It can invigorate the original respiratory function, purge the pathogenic fire, improve acuity of vision, open the specific opening of the heart, benefit intellectual power, increase the spirit, arrest convulsion, relieve thirst and restlessness, promote blood circulation, disintegrate mass and clear away phlegm.

Ageratum has the efficacy of ventilation and removing filthy substance. Pungent and sweet in taste and a little warm in property; it enters the major Yin channel of limbs. Used in cholera with vomiting and diarrhea, gripping pain of heart and abdomen, debility of the lungs with cold and the accumulation of heat in upper portion of the body cavity, it can promote functional activities, normalize the middle-abdomen (stomach and spleen), increase appetite and prevent vomiting, remove filthy substance and stimulate the appetite. It has square and nodose stems and solanum-shape leaves. Only its leaves were used as medicine in ancient times, whereas its twigs and stems are used as well as leaves in present days because of too many false leaves appearing.

This medicine is made by means of: every component is ground into powder and mixed together in weight proportion; then add an appropriate amount or water, boil by slow fire and filtrate into aqua form, or dry the filtrate into powder form, bolus form or tablet form.

The dosage is 20-100 grams per day, directed by a doctor.

This medicine belongs to a kind of pure Chinese traditional medicine biological product, without any toxicity or side effect, easy to take and having a remarkable curative effect.

This medicine can activate human immune system, promote human immune functions, accelerate human metabolism by the increase of cell division and cell regeneration. Therefore, new cells will rapidly replace the cancer cells and other dead cells so as to keep human body with normal cells and in normal operation, and finally to reach the goal of cancer prevention and treatment, youth keeping and life prolong.

The invention is further described by the following examples:

### EXAMPLE 1

Grind turtle, ageratum, ginseng and glucose into powder, mix turtle 100 g, ageratum 5 g, ginseng 3 g and glucose 5 g together, add an appropriate amount of water, boil by slow fire and filtrate into aqua form.

### EXAMPLE 2

Grind turtle, ageratum, ginseng and glucose into powder, mix turtle 200 g, ageratum 8 g, ginseng 10 g and glucose 10 g together, add an appropriate amount of water, boil by slow fire, filtrate, dry and extract into tablet form.

### EXAMPLE 3

Grind turtle, ageratum, ginseng and glucose into powder, mix turtle 100 g, ageratum 2 g, ginseng 4 g and glucose 5 g together, add an appropriate amount or water, boil by slow fire, filtrate, dry and extract into bolus form.

What is claimed is:

1. A pure biological life-prolong product comprising:
Soft-shelled turtle;
Ageratum;
Ginseng root;
Glucose;
wherein the product comprises the following ingredients in weight proportion:

| | | |
|---|---|---|
| (a) | Soft-shelled turtle | 100; |
| (b) | Ageratum | 2-5; |
| (c) | Ginseng root | 2-5; |
| (d) | Glucose | 2-5. |

2. The pure biological life-prolong product as claim 1, wherein the said soft-shelled turtle is made by selecting mature soft-shelled turtles, removing their heads and necks, keeping their bodies and tails.

3. The pure biological life-prolong product as claim 1, wherein the product is made by means of: every component is ground into powder and mixed together in weight proportion; then add an appropriate amount of water, boil by slow fire and filtrate into aqua form, or dry the filtrate into powder form, bolus form or tablet form.
